(19) United States
(12) Patent Application Publication
(10) Pub. No.: US 2006/0015151 A1
(43) Pub. Date: Jan. 19, 2006

(54) Method of using endoscopic truncal vagoscopy with gastric bypass, gastric banding and other procedures

(76) Inventor: William N. Aldrich, Napa, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/175,553

(22) Filed: Jul. 5, 2005

Related U.S. Application Data
(63) Continuation-in-part of application No. 11/067,185, filed on Feb. 24, 2005.
(63) Continuation-in-part of application No. 11/067,063, filed on Feb. 24, 2005.
(63) Continuation-in-part of application No. 10/389,236, filed on Mar. 14, 2003.

(51) Int. Cl. A61N 1/18

(52) U.S. Cl. 607/40

(57) ABSTRACT

Methods and devices for treating obesity by combining known bariatric surgeries with a method for disrupting the vagal nerve. The method for disrupting the vagal nerve comprises use of a high energy delivery device which is positioned within the esophagus to deliver transesophageal energy to interrupt the function of one or both branches of the vagal nerves. Combination of this method with known methods, such as gastric bypass and gastric banding, may increase weight loss by the patient in comparison to a single treatment on its own. In addition, methods and devices for disposing a restrictive structure such as a mesh around a selected segment of the gastrointestinal tract prevents enlargement of the segment.

### CROSS REFERENCE TO RELATED PATENT APPLICATIONS

This application is a continuation-in-part of U.S. patent application Ser. Nos. 11/067,185, titled “Methods and Apparatus for Treatment of Obesity With An Ultrasound Device Movable in Two or Three Axes,” and 11/067,063, titled “Methods and Apparatus for Testing Disruption of a Vagal Nerve,” both of which were filed on Feb. 24, 2005, and is a continuation-in-part of U.S. patent application Ser. No. 10/389,236, titled “Methods and Apparatus for Treatment of Obesity,” filed Mar. 14, 2003, all of which are incorporated by reference herein.

### FIELD OF THE INVENTION

The field of the present invention is methods and devices for treating obesity, and more particularly, methods and devices for treating obesity by combining existing treatments for obesity with methods for disrupting the vagal nerve.

### BACKGROUND OF THE INVENTION

Obesity has become an ever-increasing health problem. While such voluntary weight reduction programs as dieting and exercise have been helpful for some, many obese persons have required surgery to address their obesity problem. Two such surgical procedures are adjustable laparoscopic gastric banding (LGB) and the Roux-en-Y gastric bypass procedure. Both procedures are now well known, and each has achieved some success in reducing the weight of the patient. While these procedures have demonstrated a reasonable level of efficacy, there is a need for an improvement in the treatment of obesity that would further assist the patient to create an even more effective treatment of obesity.

### SUMMARY OF THE INVENTION

The invention is, in general, directed to the treatment of obesity by creating an interruption of the vagal nerve, preferably in the region of the esophagus, by minimally or noninvasive means. While the present invention is not to be tied to any particular theory of operation, it appears that a hunger signal is expressed by ghrelin, a peptide primarily produced in the stomach, and transmitted to the brain through the vagal nerve. The literature e.g., “The Role of the Gastric Afferent Vagal Nerve in Ghrelin-Induced Feeding and Growth Hormone Secretion in Rats,” Gastroenterology 2002:123:1120-1128 (October 2002) by Yukari Date et al. and “Gastroplasty for Obesity: Long-term Weight Loss Improved by Vagotomy,” World Journal of Surgery, Vol. 17, No. 1, January/February 1993, by Kral et al., supports this theory. The Date et al. article concluded that blockade of the gastric vagal afferent abolished ghrelin-induced feeding in rats and the Kral et al. article concluded that vagotomy combined with gastroplasty was more effective in controlling weight loss than gastroplasty alone. These articles are incorporated by reference herein.

Typically, there are two main branches, or trunks, of the vagal nerve which are located approximately 180° from each other on the outer wall of the esophagus. Depending on patient needs, it may be sufficient to interrupt only a portion of the fibers in the nerve. In this regard, it is to be noted that, in general, myelinated vagal nerve fibers, i.e., fibers that have an outer coating, are efferent. In contrast, afferent vagal nerves are unmyelinated and have no outer covering. For some patients, it may be sufficient to interrupt the function of only the afferent vagal fibers. However, the invention can be used to disrupt the vagal nerve at other locations, such as at the diaphragm.

The objective is, of course, weight loss by the patient as a result of interruption of efferent gastric and afferent hormonal signals transmitted through the vagal nerve branches. Thus, the success of the procedure described herein will, to some extent, be patient-dependent and, in some patients, it may be necessary to interrupt both the afferent and efferent vagal fibers, both of which may be found in the posterior and anterior branches.

In practicing the present invention, an energy source may be installed in the esophagus through the throat, but nasogastric access through the nose and extracorporeal application are also contemplated. The energy emitted from the energy source may be delivered to the vagal nerve through the esophagus wall, e.g., when ultrasound is used, or by causing an energy delivery device, e.g., an electrode to be passed through the wall of the esophagus.

Still other energy sources can be used to interrupt the function of the vagal nerves including thermal, microwave, laser and cryogenic energy. Alternatively, the vagal nerve function can be interrupted by transesophageal delivery of a neurotoxin such as capsaicin, atropine, or botulinum toxin. Still further, mechanical means can be used to crush the vagal nerve, e.g., with a clip or pincer, or the vagal nerve can be cut transesophageally with an appropriate cutting instrument. In a preferred embodiment of the present invention, the vagal nerve will be interrupted in the vicinity of the zig-zag line, also known as the Z-line, which is generally located in the lower esophagus between the cardiac notch of the stomach and the diaphragm.

An embodiment of the invention is directed to a method of combining the use of an endoscopic energy source in the esophagus that emits an energy source sufficient to disrupt the vagal nerve with the use of known treatments for obesity, such as gastric banding or gastric bypass. Combination of the treatments may result in more weight loss than either treatment by itself. Other example embodiments of the invention are directed to methods and devices for application of a mesh or other restrictive structure to portions of the gastrointestinal tract to maintain or confine the desired size of that portion following surgery. For example, following a gastric banding procedure, a restrictive structure may be placed around the small, upper gastric pouch to prevent pouch enlargement.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention is not limited to require the details of the example embodiments.

### DESCRIPTION OF THE DRAWINGS

The details of the invention, including fabrication, structure and operation, may be gleaned in part by study of the accompanying figures, in which like reference numerals refer to like segments. The figures are not to scale and the size of the features in relation to each other is not intended to limit the invention in any way.

FIG. 1 is a diagrammatic illustration of the general anatomy of the stomach and esophagus.

FIG. 2 illustrates positioning of an ablation device using a single balloon installed above the diaphragm.

FIG. 3 illustrates positioning the ablation device using a balloon which is inflated in the stomach.

FIG. 4 illustrates a positioning device using radially extending feet.

FIG. 5 illustrates a positioning device using a bite block.

FIG. 6 is a diagrammatic illustrate of the use of needles or electrodes to detect and ablate around the circumference of the outer surface of the esophagus in a manner designed to interrupt all vagal nerve branches.

FIG. 7 is an illustration of an ablating device which ablates a sector of the circumference of the outer wall of the esophagus.

FIG. 8 shows ablating at multiple levels.

FIG. 9 illustrates an ablation ring which can be adjusted to ablate at different angles relative to the access of the esophagus.

FIG. 10 illustrates the use of still another ablation device to locate and interrupt the vagal nerve.

FIG. 11 illustrates an endoluminal burge test which can be used to determine the extent of ablation accomplished.

FIG. 12 shows an ultrasound ablating device which may be used according to the present invention.

FIG. 13A illustrates an ultrasound device installed in the esophagus.

FIG. 13B illustrates the stomach and esophagus with an elongate device with a D-shaped distal tip.

FIG. 13C illustrates a cross section of the esophagus of FIG. 13B to show the D shaped distal tip inside the esophagus.

FIG. 14 illustrates an ablation device installed in the esophagus in a manner which shows the esophasgus held in its naturally relaxed configuration by a transducer device.

FIGS. 15 and 16 illustrate an alternative to the device shown in FIG. 14.

FIG. 17A illustrates a perspective view of a preferred embodiment of the invention when the ultrasound transducer platform is in a fully lowered position.

FIG. 17B illustrates a perspective view of a preferred embodiment of the invention when the ultrasound transducer platform is in a fully raised position.

FIG. 18 illustrates a perspective view of a preferred embodiment of the transducer platform.

FIG. 19 illustrates a perspective view of a preferred embodiment of a position actuator.

FIG. 20 illustrates an example of the focal point and distribution of energy emitted from the ultrasound transducer.

FIG. 21A illustrates the stomach and esophagus with a gastric band.

FIG. 21B illustrates an example embodiment of the invention wherein the stomach and esophagus has a gastric band and a restrictive structure is attached to a portion of the gastrointestinal tract.

FIG. 22A illustrates the stomach, esophagus and small intestine following RNY gastric bypass surgery.

FIG. 22B illustrates the stomach, esophagus and small intestine following RNY gastric bypass surgery in an example embodiment of the invention with a restrictive structure attached to a portion of the gastrointestinal tract.

### DETAILED DESCRIPTION OF THE INVENTION

Before turning to the manner in which the present invention functions, it is believed that it will be useful to briefly review the anatomy of the stomach and the esophagus. The esophagus is a muscular tube that carries food from the throat to the stomach and which passes through the diaphragm. The top end of the esophagus is the narrowest part of the entire digestive system and is encircled by a sphincter (circular muscle) that is normally closed but can open to allow the passage of food. There is a similar sphincter at the point where the esophagus enters the stomach. The walls of the esophagus consist of strong muscle fibers arranged in bundles, some circular and others longitudinal. The inner lining of the esophagus consists of smooth squamous epithelium (flattened cells).

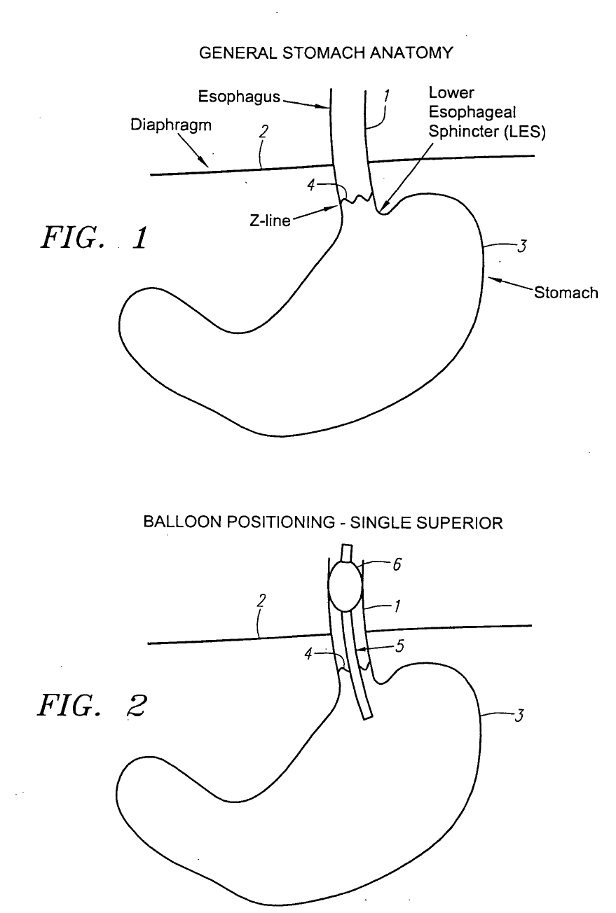

As shown in FIG. 1, the esophagus 1 extends through the diaphragm 2 into the stomach 3. Vagal nerve branches extend from the stomach along the outer wall of the esophagus to the brain. At the lower end of the esophagus, the juncture of the esophageal and gastric mucosa forms a zig-zag line 4, usually referred to as the Z-line. In the area extending from the diaphragm to a point below the Z-line, there is a subhiatal fat ring which surrounds the outer wall of the esophagus. The vagal nerve branches run between the outer wall of the esophagus and the hiatal fat ring in this area. This anatomy is well understood by those skilled in the art and a more detailed description can readily be found in a standard work such as Gray's Anatomy.

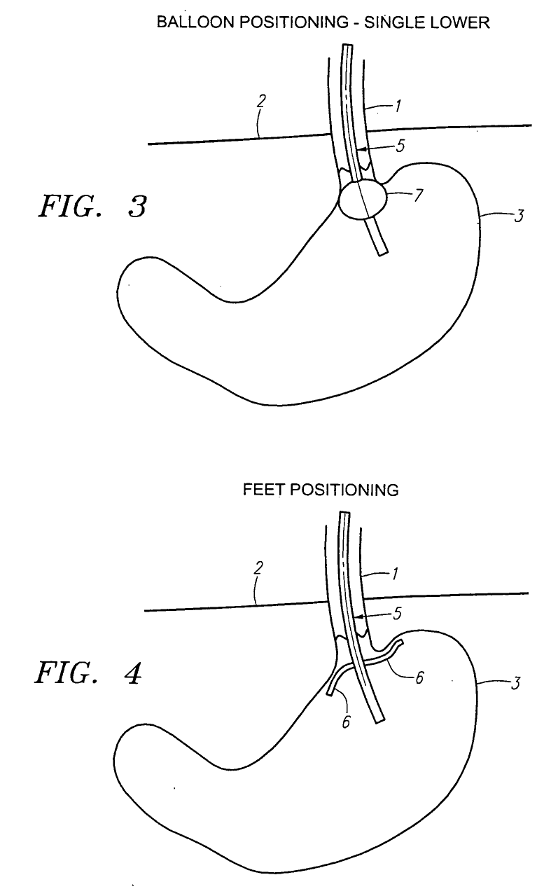

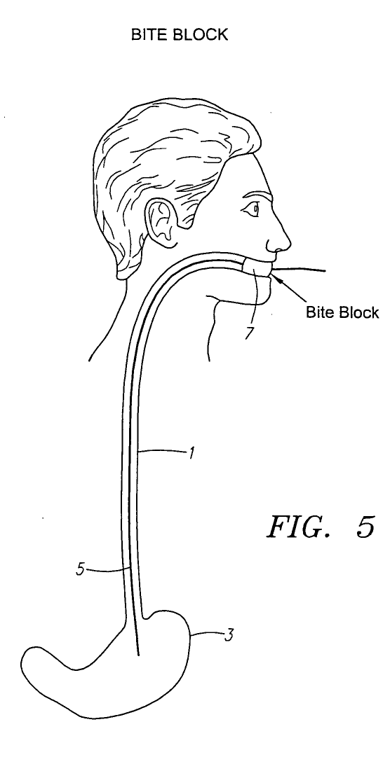

FIG. 2 illustrates in a diagrammatic manner an ablation device 5 which is held in place by balloon 6 which is inflated inside the upper portion of the esophagus. FIG. 3 illustrates positioning the ablation device 5 with balloon 7 which is inflated inside stomach 3. FIG. 4 illustrates positioning the ablation device 5 with feet 6 which pass through the esophagus folded against the ablation device 5 and then are extended inside stomach 3. FIG. 5 illustrates the use of a bite block 7 to position the ablation device 5 in stomach 3. FIG. 6 is a diagrammatic transverse cross section of the esophagus showing, in diagrammatic form, the esophagus wall 1, vagal nerve branches 8, a detection/ablation device 9 having needle probes 10. As shown, the needle probes 10 extend through the wall of the esophagus and can be used both to locate the vagus nerve and to ablate it. For detection purposes, the needle probes 10 are connected to an exterior control unit that detects and displays nerve activity in a manner well known to those skilled in the art. Once a vagal nerve is detected by a needle probe by sensing the activity of the nerve upon contact, the adjacent needle probes are energized and act in the manner of bipolar cautery probes which ablate the nerve and any other tissue between the needle probes. Preferably, the needle probes are designed in such a manner that they are held within the body of the ablation device until the device reaches its desired location. The needle probes can then be extended to penetrate the wall of the esophagus once the device has been positioned. Preferably, the needle probes are designed so that the electric current flows only at their tips so that the depth of the cautery can be focused to minimize damage to the esophagus. Cosman U.S. Pat. No. 4,565,200, Rydell U.S. Pat. No. 5,007,908, Edwards U.S. Pat. No. 5,370,675 and Edwards U.S. Pat. No. 6,129,726, each of which is incorporated by reference herein, disclose various types of electrode needle probe devices which can be used to deliver RF energy to tissue located within the body. Each of these patents discloses a device in which the needle probes are contained within the device until it has reached its desired location, at which time the needle probes are deployed to contact the tissue to which energy is to be delivered.

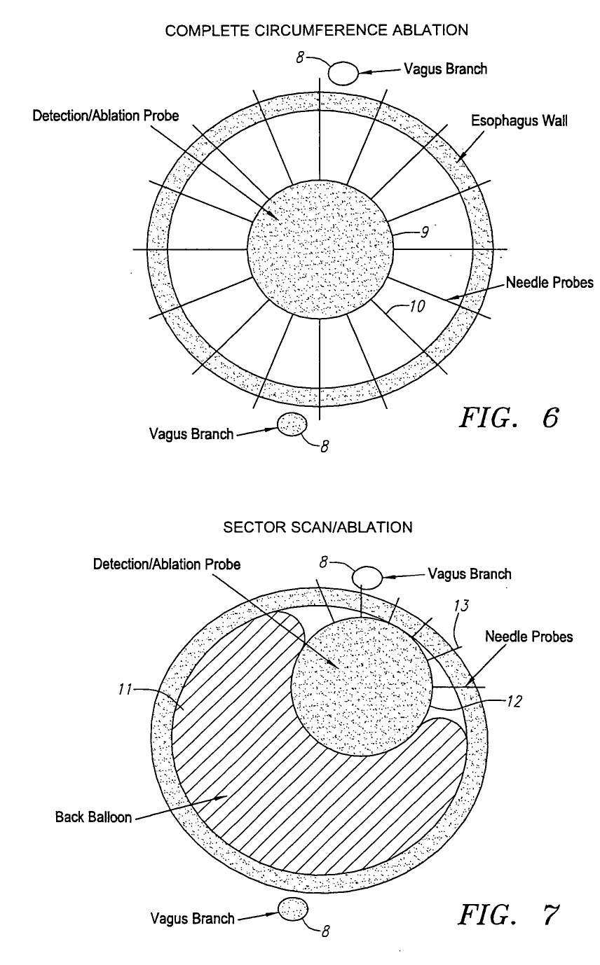

In the present invention, the needle probes can irradiate around the complete circumference of the device as shown in FIG. 6 or from only a portion of the device as shown in FIG. 7. If the latter, the device can be rotated sequentially to ensure complete coverage. As further shown in FIG. 7, when the needle probes 13 radiate from only a portion of the circumference of the device 12, a back balloon 11 can be used to position the device 12 in the desired location.

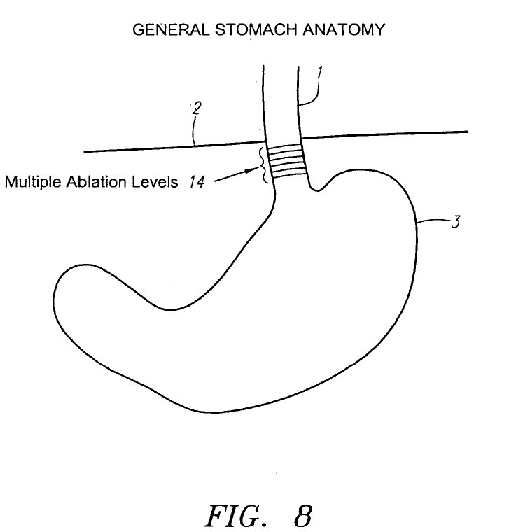

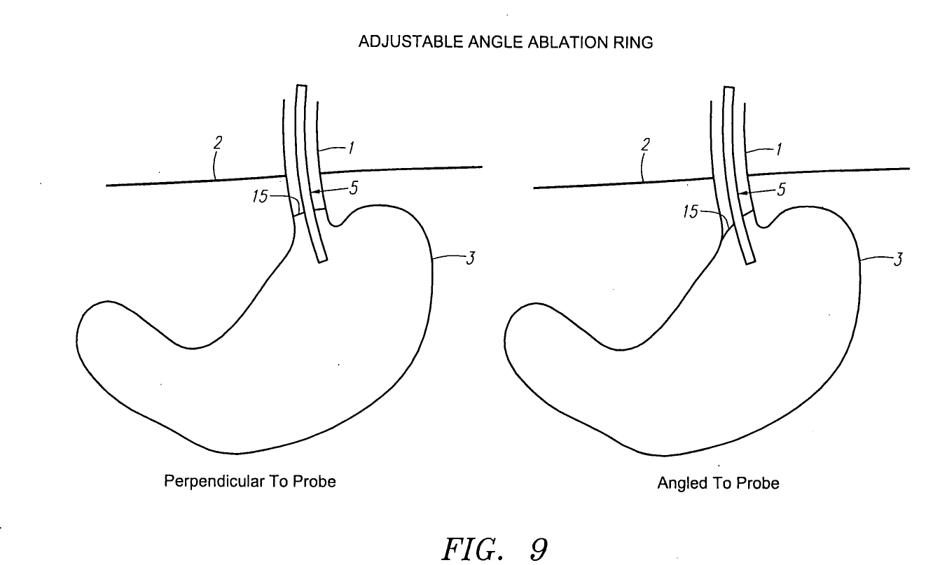

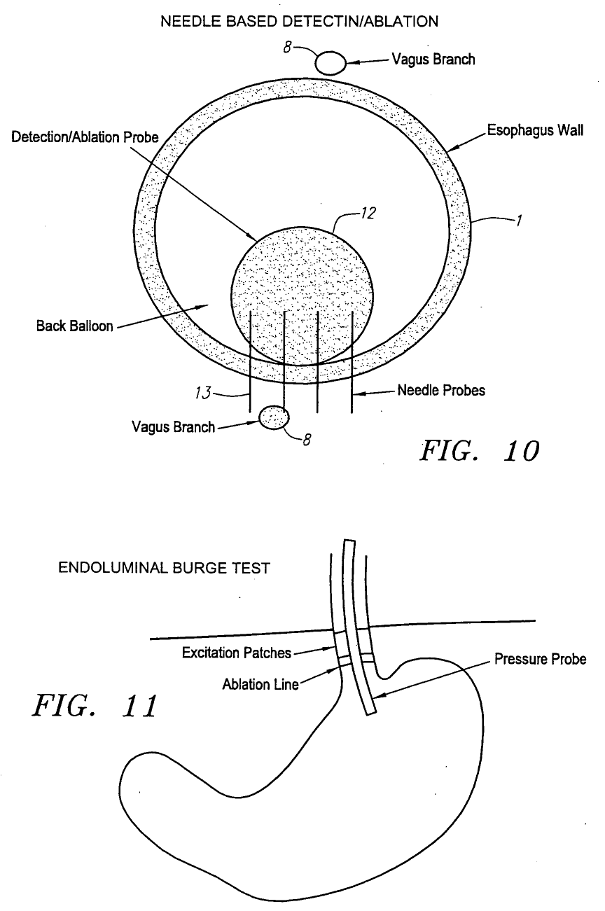

FIG. 10 illustrates an alternative sector-specific ablation device in which needle probes 13 are activated by device 12 to locate and ablate the vagal nerve in the manner described above. If a patient can obtain the desired benefit of obesity reduction by ablating the two main vagus branches 8, the procedure is simplified and the amount of ablation necessary is reduced. On the other hand, as shown in FIG. 8, if multiple ablation levels 14 are found to be necessary to provide the desired benefit to the patients, more than one ablation can be performed.

If the patient's anatomy makes it desirable, an ablation device 5 can be provided with an energy delivery component 15 which is adjustable such that energy can be delivered perpendicularly to the probe or at an angle to the probe.

When a needle probe is used to deliver energy according to the present invention, the device can be provided with temperature sensors such as thermocouples which are disposed in the distal region of the needle probes. The needle probes can be formed of a variety of materials including nickel-titanium alloy. The needle probes can assume a linear or curved shape when deployed. The device may also be provided with means for cooling the treatment site with a suitable fluid such as water, air, or other liquid or gas, to control the temperature at the treatment site. Thus, the temperature sensor can either cause a cooling medium to be provided or shut off the delivery of energy through one or more needle probes.

In a preferred embodiment of the present invention, high intensity focused ultrasound (HIFU) is used to ablate the vagal nerve branches. The HIFU energy can be transmitted transesophageally to ablate the vagal nerves on the outer wall of the esophagus.

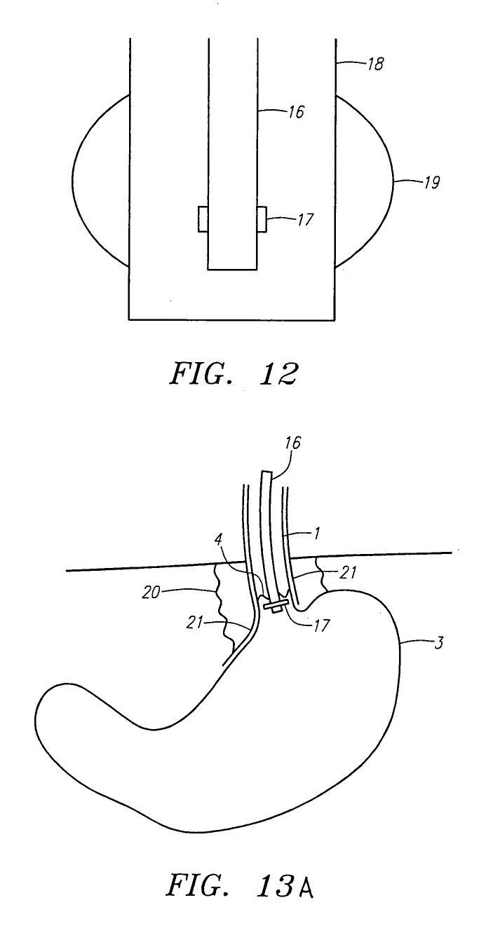

FIG. 12 illustrates in a diagrammatic form an ultrasound device which can be used according to the present invention. As shown, the device comprises an elongated member 16 which has an ultrasound transducer 17 mounted on its distal region. The elongated member is positioned in a housing 18 which is provided with an inflatable balloon 19. This device may be installed by passing it through the throat and down the esophagus until it reaches its desired location with the balloon 19 deflated. Xray, magnetic resonance imaging, or other known imaging techniques may be used to ascertain the positioning of the treatment device 50, or any other device described herein, in the gastroesophageal region, including axially down the esophagus and rotationally toward the anterior vagus nerve trunk. After rotating the treatment device 50, for example by 180 degrees to target the posterior vagus nerve trunk, the new position of the device 50 may be confirmed by xray, magnetic resonance imaging, or other known imaging techniques. The balloon 19 can then be inflated to position the device and the ultrasound transducer can be activated to transmit energy radially outwardly. Alternatively, a vacuum device can be used to position the housing.

A preferred embodiment uses an ultrasound device that is movable along up to three axes. In particular, the preferred embodiment has an ultrasound device that may be moved longitudinally along the axis of the esophagus to a further or closer distal position, transversely along the radius of the esophagus, and rotationally about the axis of the esophagus. These three degrees of freedom are relative to the esophagus. Because the ultrasound device is movable along the radial axis, the device is better able to focus its energy output on the vagal nerve in the region of the esophagus to interrupt the function of the vagal nerve, while avoiding injury to the esophagus. After an ablating or other nerve dysfunction causing device installed in the esophagus is properly positioned, it may be used to deliver ablating energy to one or more vagal nerve branches in a transesophageal manner. The anatomy of the vagal nerve complex varies somewhat from person to person, but, common to all is a structure comprising multiple vagal nerve branches located on the outer wall of the esophagus which run generally longitudinally along the esophagus wall. The preferred embodiment contemplates interrupting the function of one or more vagal nerve branches in a transesophageal manner by using various types of energy including radio frequency (RF) energy, high intensity ultrasound, high intensity focused ultrasound, and other types of energy as described in more detail in this patent specification.

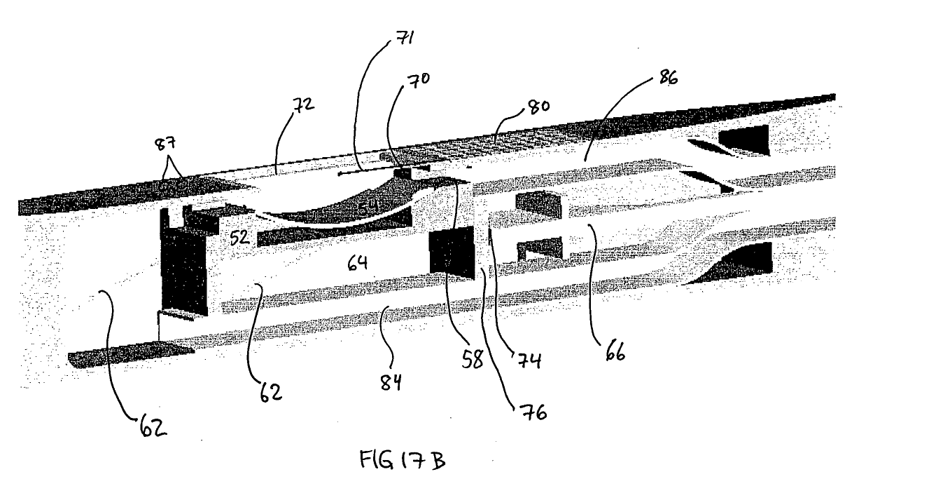

As shown in FIGS. 17A and 17B, the preferred embodiment of the present invention uses an ultrasound device 54 in a treatment device 50 that is movable along two axes. The treatment device 50 preferably treats obesity by disrupting the gastric vagal nerve adjacent the esophagus. In this example embodiment, a movable platform 52 carries a high focus ultrasound (HIFU) transducer device 54. The transducer 54 may be have an air-backing 55, or other types of known transducer backing materials. FIG. 17A illustrates a perspective view of the preferred embodiment when the platform 52 is in a fully lowered position, while FIG. 17B illustrates a perspective view when the platform 52 is in a fully raised position. Of course, the ultrasound transducer 54 may move anywhere between the fully raised position and the fully lowered position. Thus, the platform 52 may move the ultrasound transducer 54 closer to or farther from a treatment window 72 so as to control the focal point of the energy output from the ultrasound transducer 54. As the ultrasound transducer 54 moves farther from the treatment window 72, the focal point of the energy from the ultrasound transducer 54 moves closer to the wall of the esophagus. FIG. 20 illustrates an example of the focal point 90 and distribution 92 of energy emitted from the ultrasound transducer. Thus, the focal point 90 is adjustable. Preferably, the focal point 90 is directed at the site of a vagal nerve and away from the esophagus wall.

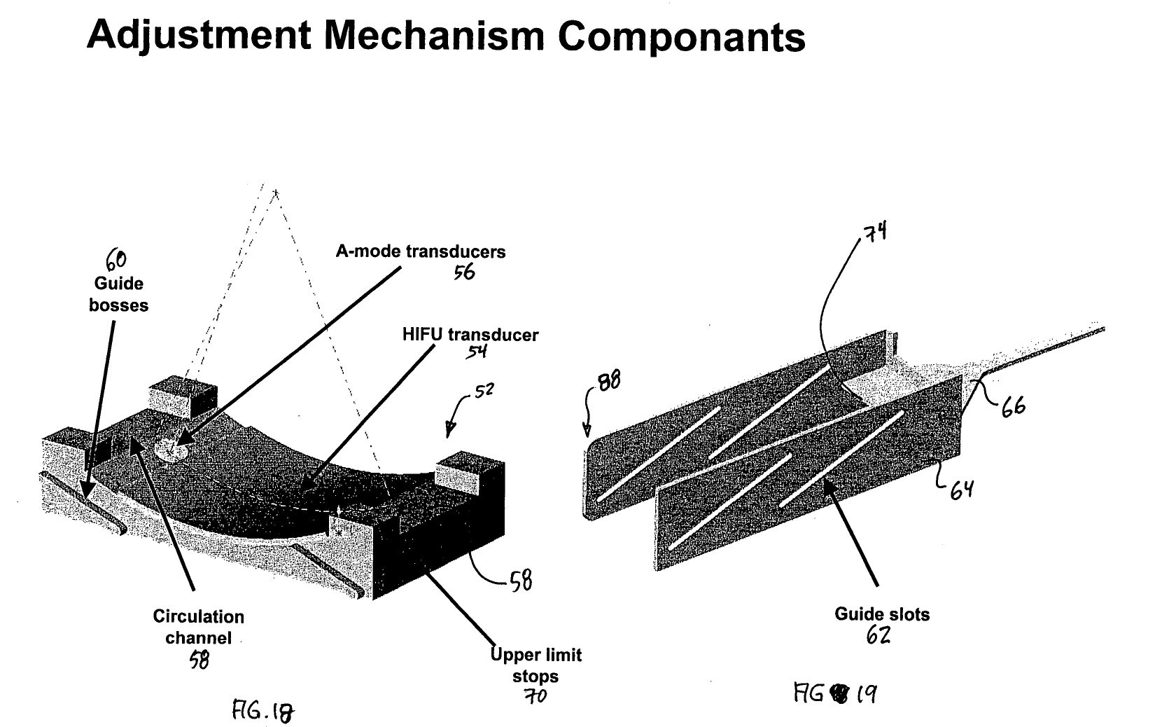

FIG. 18 illustrates a perspective view of an example embodiment of the transducer platform 52. The platform 52 preferably carries a high intensity focused ultrasound transducer 54 and an ultrasound imaging transducer 56. The ultrasound imaging transducer 56 performs diagnostic imaging for monitoring the formation of lesions in the esophagus and for defining the outside of the esophagus for the purpose of locating the vagal nerve. The ultrasound imaging transducer 56 can be any known type of imaging transducer such as those that are mechanically based (e.g., rotating and pivoting transducers) or piezo electrically based phased arrays, which have, for example, 128 imaging transducers.

The platform 52 also may include circulation channels 58 for allowing fluid, such as saline, to flow into the device and around the ultrasound transducer 54 so as to improve the acoustic characteristics of the ultrasound transducer 54 or to cool the transducer 54. Even though the transducer 54 is illustrated as having a curved surface, the ultrasound transducer 54 may have any geometry, size, shape and curvature as appropriate.

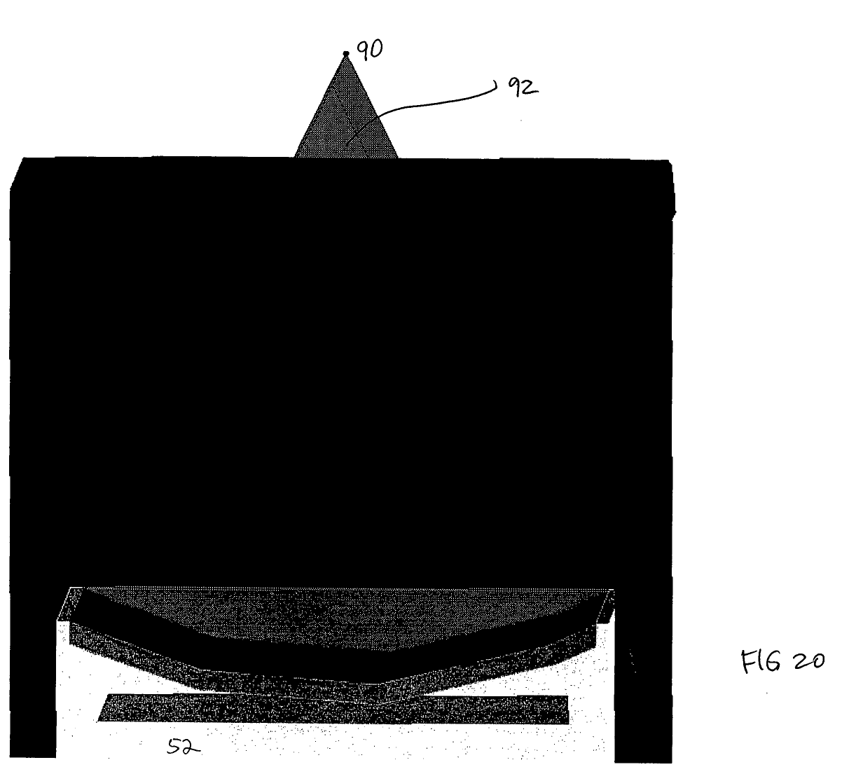

The platform 52 has one or more guide rails or guide bosses 60, which couple to guide slots 62 of the position actuator 64 shown in FIG. 19, which illustrates a perspective view of an example embodiment of the position actuator 64. Because the guide bosses 60 ride in upward slanted guide slots 62, movement of the distal end 88 of the position actuator 64 toward the platform 52 causes the platform 52 to rise toward the treatment window 72. The upper limit stops 70 on the platform 52 create an upper limit of motion for the platform 52. Of course, variations are also contemplated. For example, the guide slots 62 can be in a falling configuration so that movement of the distal end 88 of the position actuator 64 toward the platform 52 causes the platform 52 to retreat from the treatment window 72. As another example, guide bosses 60 and guide slots 62 may be replaced by any other known mechanism, such as gears, levers or a set of guide rails, to translate the platform 52 toward and away from the treatment window. The guide bosses 60 may be on two or more sides of the platform 52, which would require guide slots 62 on two or more corresponding sides of the position actuator 64. The upper limit stops 70 could hang from the inner surface of the wall having the treatment window 72 instead of being on the platform 52.

As shown in FIG. 19, the position actuator 64 has an elongate member 66 so the physician can push the actuator 64 distally or pull the actuator 64 proximally. A forward stop 74 defines the furthest distal position that the position actuator 64 may be moved.

Turning to FIG. 17A, the platform 52 is shown in its fully lowered position. As such, the forward stop 74 of the position actuator 64 is not engaged with corresponding stop 76 in treatment device 50. FIG. 17A also illustrates a nerve mapping device 80, which is preferably a 10×10 constant current impedance grid for nerve mapping. A thermocouple 71 to monitor the mucosal layer may also be provided on the outer surface of the treatment device 50.

An inflow channel 84 and outflow channel 86 may be provided so that fluids, such as saline, may flow through the treatment device 50. Additionally, optional micro holes 87 may be provided in the wall of the treatment device 50 to facilitate the flow of fluids into and out of the device 50.

Comparing FIG. 17A to FIG. 17B, one will see that the position actuator 64 in FIG. 17B is fully inserted so that the forward stop 74 has engaged corresponding stop 76, and the platform 52 is fully raised. Therefore, in this example preferred embodiment, moving the position actuator 64 distally relative to the treatment device 50 causes the platform 52 to move toward the treatment window 72. Conversely, in this example preferred embodiment, moving the position actuator 64 proximally relative to the treatment device 50 causes the platform 52 to move away from the treatment window 72. Thus, the treatment device 50 permits the position of the ultrasound transducer 54 relative to the treatment window 72, and thus, the esophageal wall, to be adjusted. The adjustable positioning of the ultrasound transducer 54 along this axial axis permits control over the focusing of the energy emitted from the ultrasound transducer 54 onto the gastric vagal nerve in the region of the esophagus while minimizing damage to or burning of the esophageal wall.

Besides translation along the axial axis, the platform 52 and ultrasound transducer 54 may be moved longitudinally along another axis to a further or closer distal position. Because the ultrasound transducer 54 can be moved longitudinally, e.g., closer or further from the stomach, the treatment device 50 can be more accurately positioned to ablate or otherwise disrupt the vagal nerve. Moreover, the treatment device 50 may be used to deliver ablating energy to one vagal nerve branch in a transesophageal manner, and then moved to another vagal nerve branch for further disruption of the vagal nerve system or for testing the completeness of the prior disruption of the vagal nerve.

A preferred method of disrupting the vagal nerves is as follows: First, a treatment device 50, or any other device described herein, is positioned at the appropriate location in the esophagus, preferably with the assistance of xray, magnetic resonance imaging, or other known imaging techniques. Such imaging techniques may be used to properly position the treatment device axially down the esophagus and rotationally toward the anterior vagus nerve trunk. Then the inner esophagus is cooled and the ablation depth is adjusted with an imaging crystal along a radial line of the esophagus. High level energy is emitted from the treatment device, such as from a HIFU transducer, to ablate and disrupt the anterior vagal nerve branch. Then the treatment device is rotated by 180 degrees to target the posterior vagus nerve trunk, where the new position of the treatment device may be confirmed by xray, magnetic resonance imaging, or other known imaging techniques. Once the new position of the treatment device is confirmed as being appropriate, the ablation depth is adjusted with an imaging crystal along a radial line of the esophagus and high level energy is emitted from the treatment device to ablate and disrupt the posterior vagal nerve branch.

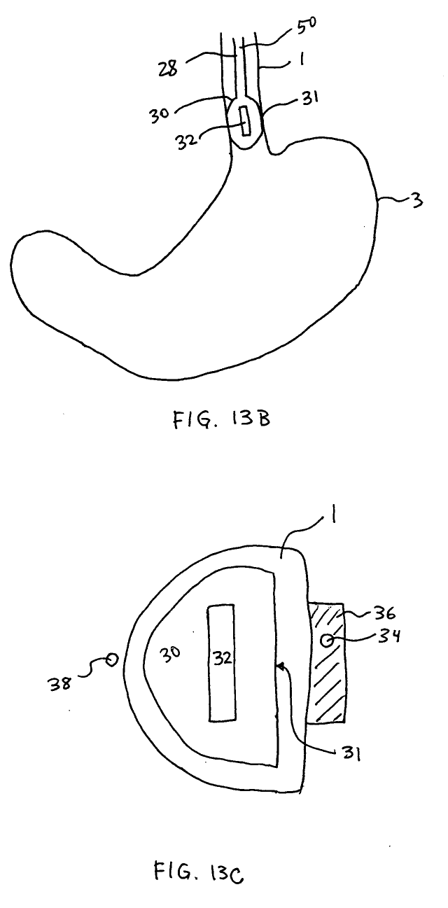

FIG. 13A is a diagrammatic illustration of an ultrasound transducer installed in the esophagus. As shown in this figure, the transducer device 16 is installed in the esophagus 1 in the region of the Z-line 4. The subhiatal fat ring 20 is also shown. When the transducer 17 is activated, ablating energy will be radiated through the wall of the esophagus to ablate the vagal nerve branches 21 which are also shown diagrammatically.

Although the esophagus is generally illustrated anatomically as a generally cylindrical tube, in its relaxed condition it assumes a more elliptical configuration which can be characterized as floppy. In other words, somewhat like a sock before it is put upon a foot, it does not assume a generally circular configuration unless it contains food or other object, but otherwise has a configuration in which the opposing walls of the esophagus are closer together than they would be when in a circular configuration. For example, FIG. 13B illustrates the stomach 3 and esophagus 1 when an elongate device 28 having a D-shaped distal tip 30 is in place in the esophagus 1. The elongate device 28 is preferably thin, flexible and torqueable. The “D” shape of the distal tip 30 causes the esophagheal wall to take on a D shape, with a flat portion 31, as further illustrated in the cross section illustration of FIG. 13C. FIG. 13C illustrates a cross section of the esophagus 1 when the D-shaped distal tip 30 is in place. A HIFU transducer 32 is preferably inside the D-shaped distal tip. By positioning the HIFU transducer 32, which is preferably directed to focus its energy at the flat portion 31 of the D, there is an ablation zone 36 that encompasses the anterior vagal nerve 34. By rotating the D-shaped distal tip 30, the ablation zone can include the posterior vagal nerve 38 or a vagal nerve branch. Thus, when the treatment device 50 is inserted into the esophagus, a cross section of the esophagus would preferably be D-shaped, where the focal point of the energy would be directed in the direction of the flat portion of the “D.”

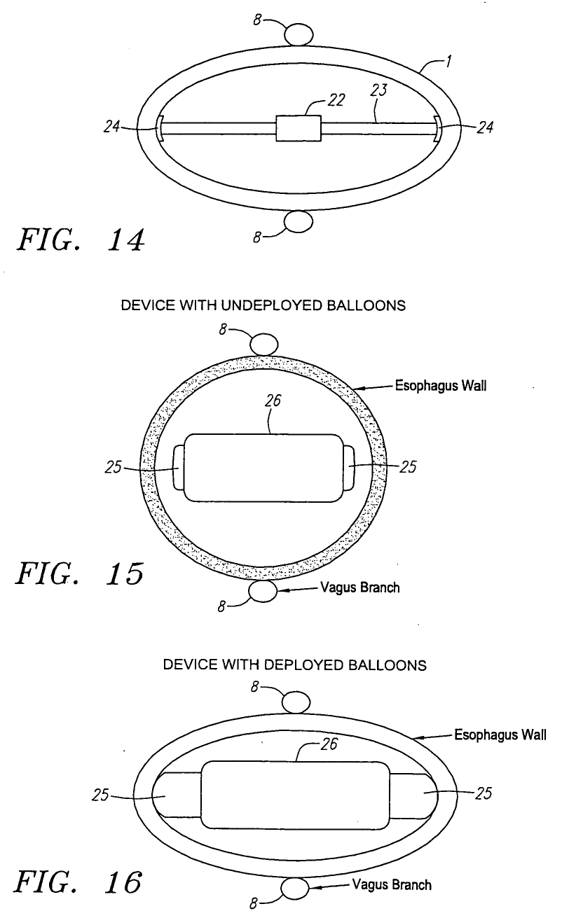

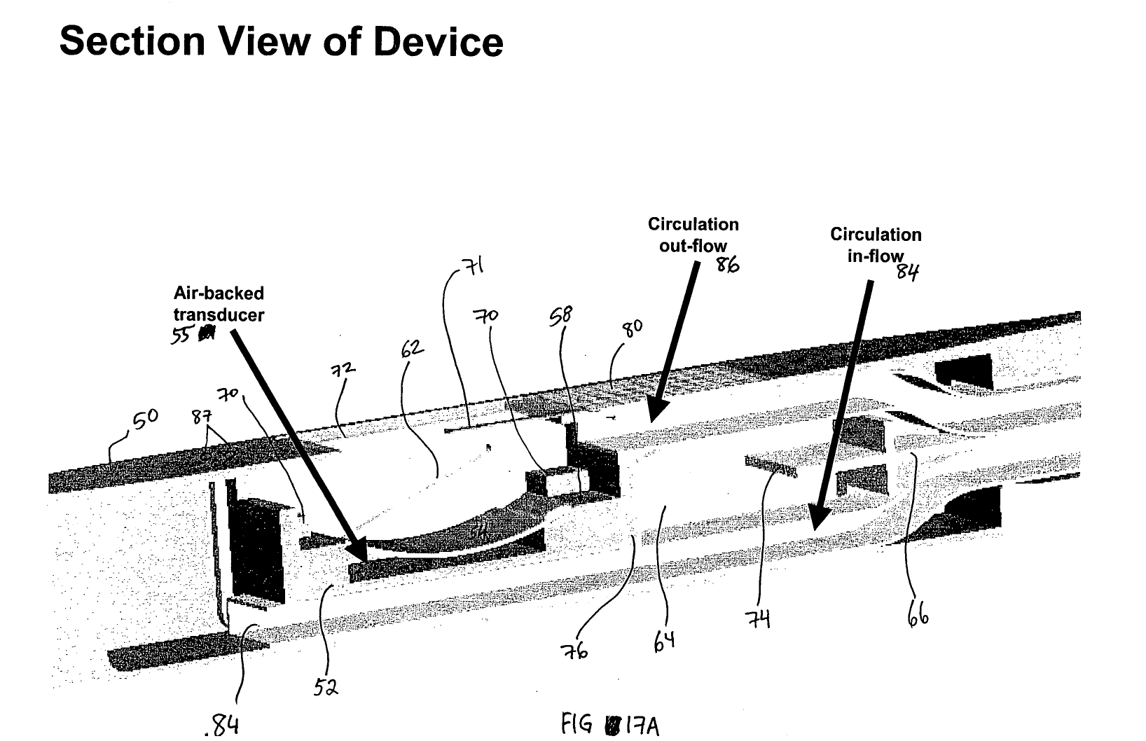

In FIG. 14, esophagus 1 with vagal nerve branches 8 on its outer wall is provided with a transducer 22 which has radially extending struts 23. Each of these struts 23 has a rounded portion 24 at its distal end. The struts 23 and 24 serve to hold the esophagus in its relaxed generally elliptical shape and to hold the transducer 22 in the desired location as well. In an alternative embodiment illustrated in FIGS. 15 and 16, balloons 25 mounted on the side of the transducer-containing device 26 are implemented to hold the esophagus in a more ellipitical shape. When these types of devices are used, the transducer device 22 or 26 could be constructed to direct ultrasound energy towards the vagal nerve branches 8 in one direction or in two directions. FIG. 15 shows the balloons 25 in the deflated state and FIG. 16 shows the balloons in the inflated state.

Ultrasound heating technology, including high-intensity ultrasound and HIFU are well understood. For example, Chapter 12, entitled “Ultrasound heating technology,” of “Thermo-radiotherapy and Thermo-chemotherapy,” vol. 1, edited by Seegenschmiedt, Fessenden and Vernon, contains a thorough explanation of the use of ultrasound in thermal therapy. This chapter is incorporated by reference herein.

The novel method of treating obesity using an endoscopic energy source in the esophagus described above, referred to hereinafter for simplicity as “endoscopic truncal vagotomy” (“ETV”), may also be combined with other known surgical procedures for the treatment of obesity. For example, ETV may be used in combination with adjustable laparoscopic gastric banding (LGB), gastric bypass surgery, and other restrictive and/or malabsortive approaches known in the art. The LGB may be either vertical or horizontal as disclosed in U.S. Pat. No. 6,773,440, the disclosure of which is incorporated by reference herein.

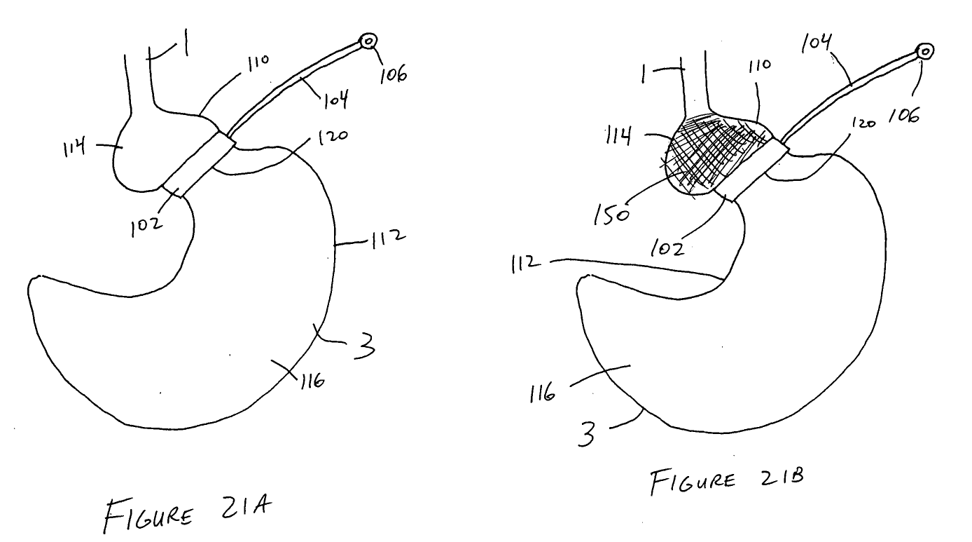

LGB is a minimally invasive surgical technique for the treatment of morbid obesity. LGB is designed for long-term weight loss by restricting the size of a one's stomach without the need to cut either the stomach or intestines. As shown in FIG. 21A, the procedure involves placing a band 102 around an upper portion of the stomach to divide the stomach into a first region 110 and a second region 112. The first region comprises a small, upper pouch 114, and the second region comprises a larger, lower pouch 116. The capacity of the upper pouch is typically approximately 15 cc but can be a different volume. The small capacity of upper pouch 114 reduces the amount of food that a patient can eat, and the patient can feel full after eating smaller portions. Band 102 creates a channel, or stoma 120, of reduced circumference between the upper and lower pouches 114, 116 that decreases the speed of food as it passes between the two pouches thereby allowing the patient to feel full longer. Typically a silicon-elastomer band is used, the diameter of which can be adjusted after the initial surgery based on the progress and needs of the patient. In some embodiments, injection or removal of saline in the band 102 results in decrease or increase of the diameter of the band, respectively. In this embodiment, a tube 104 connects the band 102 to a reservoir 106 placed beneath the skin. A medical provider can then inject or remove saline from the reservoir 106 using a needle. When LGB is combined with applicant's ETV procedure, a patient may experience an even greater ability to lose weight compared to either procedure by itself.

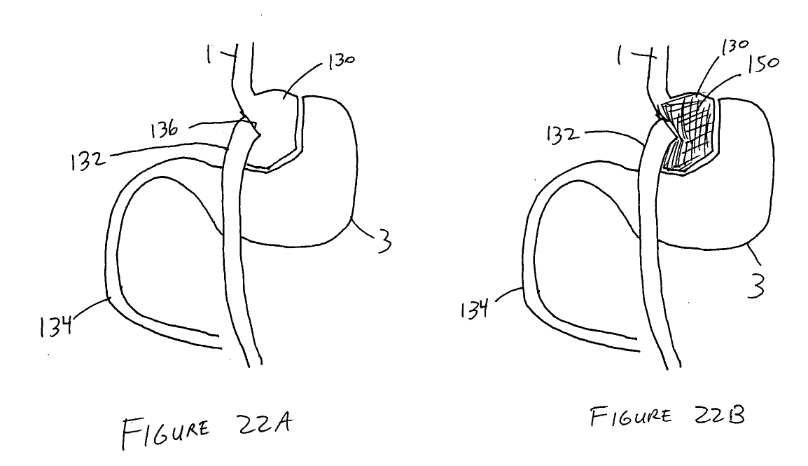

Applicant's ETV procedure may also be combined with gastric bypass surgery to increase the weight loss of the patient. There are several forms of gastric bypass surgery. One of the most common types is Roux-en-Y (RNY) gastric bypass surgery. As illustrated in FIG. 22A, RNY is a malabsorptive and restrictive technique that involves the stapling of the stomach 3 to create a small pouch 130 that is able to hold only a small amount of food. Generally the size of the pouch 130 is between 15 and 20 cc, but the volume may be otherwise. In this procedure, a portion of the small intestine typically from the second portion of the small intestine, or jejunum 132, is shaped in the form of a “Y” and then brought up and connected to this small pouch 130. This connection results in the bypassing of the main portion of the stomach and the first portion of the small intestine or duodenum 134, as well as a portion of the jejunum. The stomach outlet 136 is small so that food leaves the stomach pouch 130 slowly, which leaves the patient feeling full longer.

The ETV procedure may also be combined with other surgical procedures that reduce the size of the stomach and/or alter the configuration of the gastrointestinal tissue such as the methods disclosed in U.S. Patent Application Publication Nos. 20040147958 (Lam et al.) and 20040122453 (Deem et al.), each of which is incorporated herein by reference in its entirety. Methods to insert gastric and/or intestinal sleeves such as those described in U.S. Patent Application Publication Nos. 20050049718 (Mitchell et al.) and 20050080395 (Levine et al.), each of which is incorporated herein by reference in its entirety, may also be used in combination with the ETV procedure. Other forms of bariatric surgeries such as biliopancreatic diversion, vertical banded gastroplasty, silicone ring gastroplasty, jejuno-ileal bypass, jejuno-colic shunt, and other bariatric procedures known in the art may also be combined with the ETV procedure disclosed herein. The result of the combined procedures may result in more weight control or weight loss for the patient than a single treatment on its own.

The method of the present invention may also be combined with techniques for the electrical stimulation of the vagal nerve to treat obesity such as those disclosed in U.S. Patent Application Publication Nos. 20040039427 (Barrett et al.) and 20050038484 (Knudson et al.), each of which is incorporated by reference herein in its entirety. Electrical stimulation of the vagal nerve after the ETV procedure can be used to detect whether the all desired branches of the vagal nerve have been transected. If electrical stimulation indicates that the vagal nerve has not been transected properly, a second ETV procedure may be desired. Alternatively, if one or both branches of the vagal nerve or smaller vagal nerves remain intact, electrical stimulation of the vagal nerve may itself be used to provide further treat the patient in accordance with methods known in the art.

In a further method of treatment, a restrictive structure such as a mesh may be disposed around the small pouch, and/or the large pouch, created by the RNY procedure, LGB procedure, or other procedure which creates a stomach pouch with a reduced size. The restrictive structure includes restrictive materials and may be comprised of cloth, fabric, nitinol or other restrictive members known in the art. The restrictive structure is configured to retain the desired size of the smaller pouch and prevent pouch enlargement after the surgery. As shown in FIG. 21B, for LGB procedures, a restrictive structure 150 can be placed around the upper pouch 114 above the gastric band 102 to retain the desired size of the upper pouch 114. For gastric bypass surgeries, a restrictive structure 150 may be placed around the pouch 130 as illustrated in FIG. 22B. A restrictive structure 150 may also be placed around other areas of the gastrointestinal tract as necessary to maintain the desired size of the tract.

Other obesity treatments with which ETV may be combined include tissue approximation and fixation as disclosed in U.S. Pat. Nos. 6,773,440; 6,746,460; 6,656,194 and 6,558,400, the disclosures of which are incorporated by reference herein. In addition, ETV treatment may be combined with gastrointestinal pacemaking, such as that disclosed in U.S. Pat. No. 5,292,344, the disclosure of which is incorporated by reference herein.

The sequence and timing of the combined ETV treatment and the additional interventional treatment may be varied according to patient needs. Thus, the ETV treatment and the additional interventional treatment may be performed essentially continuously and in either order, i.e., ETV first or ETV second. In addition, some time may be allowed to pass between the two treatments in order to allow for patient recovery from the first treatment or for other medical reasons.

Thus, whether ETV treatment is viewed as a supplement to other interventional treatments or other interventional treatments are viewed as a supplement to ETV treatment, the combined effect of the two treatments may be of greater benefit to the patient than, for example, performing ETV treatment or some other interventional treatment for a second time. Furthermore, the combination of other interventional treatment with ETV treatment may lead to some modification of the other interventional treatment. The present invention is to be understood to cover other interventional treatments that presently exist, in both their present and modified form, and other interventional treatments for obesity that are developed in the future. In the foregoing specification, the invention has been described with reference to specific embodiments thereof. It will, however, be evident that various modifications and changes may be made thereto without departing from the broader spirit and scope of the invention. For example, each feature of one embodiment can be mixed and matched with other features shown in other embodiments. As another example, the order of steps of method embodiments may be changed. Features and processes known to those of ordinary skill may similarly be incorporated as desired. Additionally and obviously, features may be added or subtracted as desired. Accordingly, the invention is not to be restricted, but rather to be given the full scope of the attached claims and their equivalents.

We claim:

1. A method for reducing obesity comprising:
positioning an energy delivery device within the esophagus;
energizing the delivery device to deliver energy to the site of at least one vagal nerve branch on the outer wall of the esophagus; and
performing a gastric bypass surgery.

2. The method of claim 1, further comprising attaching a restrictive structure to a portion of the stomach to maintain a desired size of the portion.

3. The method of claim 1, wherein the restrictive structure comprises a mesh.

4. The method of claim 1, wherein the restrictive structure includes cloth.

5. The method of claim 1, wherein the restrictive structure includes nitinol.

6. The method of claim 1, further comprising adjusting the position of the energy delivery device along a first axis and a second axis, the first axis being a longitudinal axis of the esophagus and the second axis being transversely along a radius of the esophagus.

7. A method for reducing obesity comprising:
positioning an energy delivery device within the esophagus;
energizing the delivery device to deliver energy to the site of at least one vagal nerve branch on the outer wall of the esophagus; and
performing a gastric banding surgery, wherein the stomach is divided into at least a small upper pouch and a larger lower pouch.

8. The method of claim 7, further comprising attaching a restrictive structure to the upper pouch configured to prevent enlargement of the pouch.

9. The method of claim 7, further comprising attaching a restrictive structure to the lower pouch configured to prevent enlargement of the pouch.

10. The method of claim 7, wherein said energy is highly focused ultrasound.

11. The method of claim 8, wherein the restrictive structure comprises a mesh.

12. The method of claim 8, wherein the restrictive structure includes cloth.

13. The method of claim 8, wherein the restrictive structure includes nitinol.

14. The method of claim 9, wherein the restrictive structure comprises a mesh.

15. The method of claim 9, wherein the restrictive structure includes cloth.

16. The method of claim 9, wherein the restrictive structure includes nitinol.

17. The method of claim 7, further comprising adjusting the position of the energy delivery device along a first axis and a second axis, the first axis being a longitudinal axis of the esophagus and the second axis being transversely along a radius of the esophagus.

18. A method for reducing obesity comprising:
positioning an energy delivery device within the esophagus;
energizing the delivery device to deliver energy to the site of at least one vagal nerve branch on the outer wall of the esophagus; and
electrically stimulating the vagal nerve with another device.

19. The method of claim 18, further comprising adjusting the position of the energy delivery device along a first axis and a second axis, the first axis being a longitudinal axis of the esophagus and the second axis being transversely along a radius of the esophagus.

20. A method for reducing obesity comprising:
positioning an energy delivery device within the esophagus;
energizing the delivery device to deliver energy to the site of at least one vagal nerve branch on the outer wall of the esophagus; and
performing surgical reduction of the size of the stomach.

21. The method of claim 20, further comprising adjusting the position of the energy delivery device along a first axis and a second axis, the first axis being a longitudinal axis of the esophagus and the second axis being transversely along a radius of the esophagus.

22. The method of claim 20, further comprising attaching a restrictive structure to the stomach configured to prevent enlargement of the stomach.

23. The method of claim 22, wherein the restrictive structure comprises a mesh.

24. The method of claim 22, wherein the restrictive structure includes cloth.

25. The method of claim 22, wherein the restrictive structure includes nitinol.

26. A method for reducing obesity comprising:
positioning an energy delivery device within the esophagus;
adjusting the position of the energy delivery device along a first axis and a second axis, the first axis being a longitudinal axis of the esophagus and the second axis being tranversely along a radius of the esophagus; and
causing the energy delivery device to deliver energy to the site of at least one vagal nerve branch on the outer wall of the esophagus.

27. The method of claim 26, further comprising creating gastrointestinal folds within the gastrointestinal tract to reduce adsorption.

28. The method of claim 26, further comprising inserting one or more gastric sleeves within the stomach.

27. The method of claim 26, further comprising inserting one or more intestinal sleeves within the small bowel.

28. A method for reducing obesity comprising:
bypassing a first region of the stomach and a first portion of the small intestine;
attaching a second portion of the small intestine directly to a second region of the stomach;
attaching a restrictive structure around the second region configured to maintain the size of the second region.

29. The method of claim 28, wherein the restrictive structure comprises a mesh.

30. The method of claim 28, wherein the restrictive structure includes cloth.

31. The method of claim 28, wherein the restrictive structure includes nitinol.

32. A method for reducing obesity comprising:
performing a gastric bypass surgery; and
attaching a restrictive structure around the stomach configured to prevent enlargement of the stomach.

33. The method of claim 32, wherein the restrictive structure comprises a mesh.

34. The method of claim 32, wherein the restrictive structure includes cloth.

35. The method of claim 33, wherein the restrictive structure includes nitinol.

36. A method of reducing obesity comprising:
attaching a gastric band around an upper portion of the stomach thereby creating at least a small upper pouch and a larger lower pouch; and
disposing a restrictive structure around the upper pouch configured to prevent enlargement of the upper pouch.

37. The method of claim 36, wherein the restrictive structure comprises a mesh.

38. The method of claim 36, wherein the restrictive structure includes cloth.

39. The method of claim 36, wherein the restrictive structure includes nitinol.

40. A method of reducing obesity comprising:
positioning an elongate body within the esophagus, the elongate body comprising an ablation device;
adjusting the position of the ablation device to a position near the site of a first vagal nerve branch disposed on the outer wall of the esophagus;
ablating the first vagal nerve with the with the ablation device.

41. The method of claim 40, further comprising performing biliopancreatic diversion surgery.

42. The method of claim 40, further comprising performing a vertical banded gastroplasty surgery.

43. The method of claim 40, further comprising performing a silicone ring gastroplasty surgery.

44. The method of claim 40, further comprising performing a jejuno-ileal bypass surgery.

45. The method of claim 40, further comprising performing a procedure to create a jejuno-colic shunt.

46. A method for reducing obesity comprising:
positioning a highly focused ultrasound delivery device within the esophagus;
energizing the device to focus on energy at the site of at least one vagal nerve on the outer wall of the esophagus to disrupt the function of said nerve; and
performing an additional interventional obesity treatment therapy.

47. The method of claim 46, wherein said additional treatment is gastric bypass surgery.

48. The method of claim 46, wherein said additional treatment is gastric banding surgery.

49. The method of claim 46, wherein said additional treatment is gastrointestinal pacing.

50. The method of claim 46, wherein said additional treatment is electrical stimulation of the vagal nerve.
